(19) United States
(12) Patent Application Publication
(10) Pub. No.: US 2025/0014757 A1
(43) Pub. Date: Jan. 9, 2025

(54) MEDICAL INFORMATION PROCESSING APPARATUS AND NON-TRANSITORY COMPUTER READABLE MEDIUM

(71) Applicant: FUJIFILM Corporation, Tokyo (JP)

(72) Inventors: Yuya YOKOYAMA, Tokyo (JP); Jun SHINGU, Tokyo (JP)

(73) Assignee: FUJIFILM Corporation, Tokyo (JP)

(21) Appl. No.: 18/764,334

(22) Filed: Jul. 4, 2024

(30) Foreign Application Priority Data
Jul. 6, 2023 (JP) 2023-111648

(51) Int. Cl. G16H 50/30; G16H 10/60; G16H 15/00

(52) U.S. Cl. CPC G16H 50/30; G16H 10/60; G16H 15/00

(57) ABSTRACT

A processor is provided, in which the processor is configured to generate a first distribution based on an age and an evaluation value related to health data, set a plurality of regions and a sentence T corresponding to each of the regions in the first distribution, acquire a measurement result including the age and the evaluation value of a user, extract a second distribution including a specific region, which is the region corresponding to the measurement result, from the first distribution, and display the second distribution, the sentence corresponding to the specific region, and the measurement result in the same display region.

### CROSS-REFERENCE TO RELATED APPLICATIONS

This application claims priority under 35 U.S.C § 119 (a) to Japanese Patent Application No. 2023-111648 filed on 6 Jul. 2023. The above application is hereby expressly incorporated by reference, in its entirety, into the present application.

### BACKGROUND OF THE INVENTION

### 1. Field of the Invention

The present invention relates to a medical information processing apparatus and a non-transitory computer readable medium.

### 2. Description of the Related Art

In a medical examination such as a health checkup, a medical institution evaluates measured health data of a medical examinee and an age thereof and creates a report provided to the medical examinee. For the evaluation of the health data, distribution data is used in which the evaluation of the health data for each age is calculated with a large amount of ages and health data, which are acquired in advance from medical examinees of a wide age group. The distribution data is created, for example, as a graph having an axis of age and an axis of measurement value or evaluation value of the health data.

In JP2005-000565A (corresponding to US2004/254496A1), graph regions are created in which two types of biological information such as a body fat percentage and a muscle mass acquired from a significant number of subjects are used as a vertical axis and a horizontal axis, the graph regions being divided according to values of the respective axes. A smoothing curve related to the two types of biological information based on age information is created on the graph regions. In the graph region, a measurement result of a user is plotted, and a comment according to the biological information and an age of the user is displayed in accordance with a position of the plotted graph region. In JP2005-092853A, a distribution of category data parted into a plurality of stages for every five years of age, which is created using a large amount of health checkup data subjected to statistical analysis processing, is displayed to evaluate clinical examination data of a medical examinee.

### SUMMARY OF THE INVENTION

On the other hand, for provision data such as a report, it takes time and effort to summarize a doctor's comment together with a measurement result evaluation. In JP2005-000565A, a comment is stored in each of nine graph regions divided into each age group. In JP2005-092853A, dividing into the category data for every five years of age, which is parted into categories of seven stages, is performed, but the comment is not assumed to be stored.

In addition to the above-described points, it is desirable to create the provision data, which includes the doctor's comment based on a relationship between the age and the health data to the measurement result of the medical examination, in about one month from an examination date, and measurement results of a large number of medical examinees are acquired in the health checkup. Thus, each piece of provision data is required to be created easily.

An object of the present invention is to provide a medical information processing apparatus and a non-transitory computer readable medium that support creation of provision data having a measurement result and a comment on the measurement result according to an age of a medical examinee.

A medical information processing apparatus according to an aspect of the present invention comprises a processor, in which the processor is configured to generate a first distribution based on an age and an evaluation value related to health data, set a plurality of regions and a sentence corresponding to each of the regions in the first distribution, acquire a measurement result including the age and the evaluation value of a user, extract a second distribution including a specific region, which is the region corresponding to the measurement result, from the first distribution, and display the second distribution, the sentence corresponding to the specific region, and the measurement result in the same display region.

It is preferable that the processor is configured to calculate the evaluation value from the health data evaluated in stages according to the age, and generate, as the first distribution, a graph in which one axis is an age axis indicating the age and the other axis is a score ratio axis indicating a ratio of the evaluation value.

It is preferable that the processor is configured to decide an age spacing, which is a scale spacing on the age axis, based on the number of the evaluation values in the first distribution.

It is preferable that the processor is configured to set the region in the first distribution based on the evaluation value and an age range in which the age axis is divided at a specific portion.

It is preferable that the specific portion is a portion where an amount of change in a specific evaluation value is largest in ratios between two consecutive evaluation values.

It is preferable that the processor is configured to display the display region in which the measurement result is displayed on the specific region in a superimposed manner or the specific region is displayed differently from another region.

It is preferable that the processor is configured to decide the sentence and a size of the second distribution to be displayed in the display region, based on the specific region.

It is preferable that the processor is configured to receive addition of the age and the health data, and update the setting of each of the regions in the first distribution based on the addition.

It is preferable that the processor is configured to perform display indicating a portion of interest in the first distribution updated based on the addition.

It is preferable that the processor is configured to reset the sentence corresponding to each of the regions updated based on the addition.

It is preferable that the processor is configured to reuse, for the region having the same feature as before update, the sentence before the update in the sentence resetting.

It is preferable that the processor is configured to adjust, in the first distribution, the age spacing in a case where the number of the evaluation values is less than a certain number.

It is preferable that the processor is configured to round, in a case where the number of the evaluation values in a scale of an end portion on the age axis is less than a certain number, a numerical value of the scale of the end portion in the first distribution.

It is preferable that the processor is configured to properly use, in the setting of the sentence, a first sentence automatically set for each specific region, a second sentence set by a user input, and a third sentence combined from the first sentence and the second sentence.

A non-transitory computer readable medium storing a computer-executable program according to an aspect of the present invention causes a computer to realize a function of generating a first distribution based on an age and an evaluation value related to health data, a function of setting a plurality of regions and a sentence corresponding to each of the regions in the first distribution, a function of acquiring a measurement result including the age and the evaluation value of a user, a function of extracting a second distribution including a specific region, which is the region corresponding to the measurement result, from the first distribution, and a function of displaying the second distribution, the sentence corresponding to the specific region, and the measurement result in the same display region.

According to the present invention, it is possible to support the creation of provision data having the measurement result and the comment on the measurement result according to the age of the medical examinee.

### BRIEF DESCRIPTION OF THE DRAWINGS

FIG. 1 is a schematic diagram of a medical information processing system.

FIG. 2 is a block diagram showing a function of a medical information processing apparatus.

FIG. 3 is a table for describing evaluation value data based on an age and health data recognized by the medical information processing apparatus.

FIG. 4 is a graph for describing a first distribution generated by the medical information processing apparatus.

FIG. 5 is a diagram for describing storage of each region set in the first distribution as a region data group.

FIG. 6 is a graph for describing setting of sentences corresponding to a first region in the first distribution.

FIG. 7 is a graph for describing setting of sentences corresponding to a sixth region in the first distribution.

FIG. 8 is a graph for describing setting of sentences corresponding to a tenth region in the first distribution.

FIG. 9 is a graph for describing setting of sentences corresponding to a fifteenth region in the first distribution.

FIG. 10 is a graph for describing setting of sentences corresponding to a sixteenth region in the first distribution.

FIG. 11 is a diagram for describing combining, regarding a ninth region, of a sentence that is automatically generated and a sentence that is manually input.

FIG. 12 is a diagram for describing combining, regarding a tenth region, of the sentence that is automatically generated and a sentence that is manually input.

FIG. 13 is an explanatory drawing including (A) a graph for describing the first distribution in which a specific region is discriminated according to a measurement result, and (B) a graph for describing a second distribution extracted from the first distribution based on the specific region.

FIG. 14 is a diagram for describing creation of a report having the second distribution including the specific region and sentences corresponding to the specific region.

FIG. 15 is a flowchart showing a series of flows of creating provision data for acquired age and health data.

FIG. 16 is an explanatory drawing including (A) a graph for describing a first distribution different from the first distribution in FIG. 4, and (B) a graph for describing a first distribution in which a score ratio changes due to additional data.

FIG. 17 is an explanatory drawing in a second embodiment, including (A) a graph for describing a first distribution in which an evaluation value is insufficient in a specific age group, and (B) a graph for describing a first distribution in which an age spacing displayed according to the age group having insufficient evaluation value is adjusted.

FIG. 18 is an explanatory drawing in a second embodiment, including (A) a graph for describing a first distribution in which evaluation values are insufficient for a certain age or younger and for a certain age or older, and (B) a graph for describing a first distribution in which a numerical value of an age range displayed according to an insufficient range is rounded.

### DESCRIPTION OF THE PREFERRED EMBODIMENTS

### First Embodiment

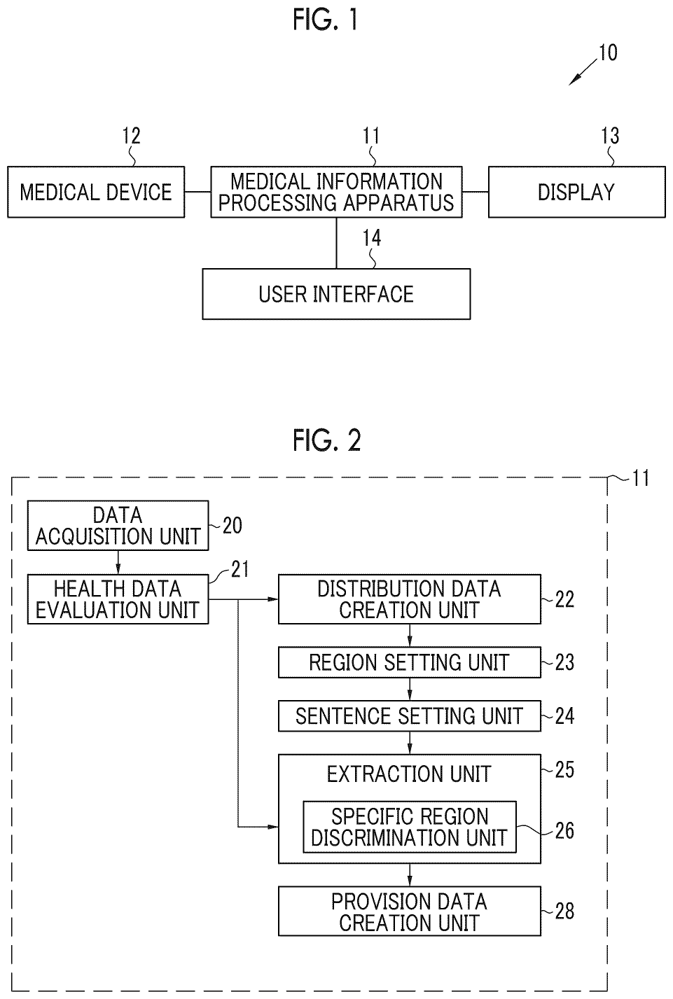

As shown in FIG. 1, a medical information processing system 10 is configured of a medical information processing apparatus 11, a medical device 12 that transmits data to the medical information processing apparatus 11, a display 13 that displays data included in the medical information processing apparatus 11, and a user interface (UI) 14 that delivers an input received from a user, such as a doctor, to the medical information processing apparatus 11. The display 13 and the user interface 14 may be provided in the medical information processing apparatus 11 instead of a connection apparatus.

The medical information processing apparatus 11 is an apparatus that comprises a storage medium and a processor and can perform processing of medical information and transmission and reception thereof, and creates, using ages and evaluation values based on health data of a large number of medical examinees, a distribution of the ages and the evaluation values.

The medical device 12 is a device that provides the medical information of a large number of medical examinees, which is collected by a medical institution through a medical examination such as a health checkup, to the medical information processing apparatus 11, and is, for example, a device that performs the medical examination or a server or database owned by the medical institution.

As shown in FIG. 2, functions of a data acquisition unit 20, a health data evaluation unit 21, a distribution data creation unit 22, a region setting unit 23, a sentence setting unit 24, an extraction unit 25, a provision data creation unit 28, an input reception unit (not shown), an output control unit (not shown), and a storage unit (not shown) are realized in the medical information processing apparatus 11. Further, the extraction unit 25 has a function of a specific region discrimination unit 26. The input reception unit (not shown) receives an input operation of the user who is a medical worker, such as a doctor, via the user interface 14 such as a mouse operation or a keyboard operation. Further, the output control unit (not shown) outputs created provision data to another device or a network.

The medical information processing apparatus 11 is a computer, such as a personal computer or a workstation, in which an application program for realizing a predetermined function is installed. The computer is provided with a central processing unit (CPU) which is a processor, a memory, a storage, and the like, and realizes various functions by a program or the like stored in the storage.

With the above functions, the medical information processing apparatus 11 easily creates, in a setting mode, the provision data obtained by summarizing a measurement result of a specific user, who is a medical examinee of the medical examination such as the health checkup, and a sentence for the measurement result into one, and provides the created provision data to the medical examinee. Distribution data used to create the sentence for the measurement result is defined as a first distribution, and the distribution data listed in the provision data is defined as a second distribution. The medical information processing apparatus switches between the setting mode, a report creation mode, and a data addition mode to perform creation of the first distribution, creation of the provision data having the second distribution, and update of the first distribution.

In the setting mode, the first distribution is generated based on the ages and the evaluation values related to the health data of a large number of medical examinees. Further, the first distribution is parted into a plurality of regions, and a corresponding sentence is set for each region.

In the report creation mode, the measurement result including the age and the evaluation value of the specific user is acquired, and the second distribution including a specific region that is a region corresponding to the measurement result, is extracted from the first distribution. The extracted second distribution, the sentence corresponding to the specific region, and the measurement result are displayed in the same display region. Further, the provision data such as a report is created with a content displayed in the display region.

In the data addition mode, the addition of the age and the evaluation value related to the health data is received, and the setting for each region in the first distribution or the sentence corresponding to each region is updated based on the addition.

The data acquisition unit 20 acquires the medical information including at least the age and the health data of the medical examinee who has undergone the medical examination from the medical device 12. In the setting mode, the medical information including the ages and the health data of a sufficient number of people, for example, 10,000 persons from teens to eighties, is collected to create the first distribution. In the report creation mode, the medical information including at least the age and the health data of the user who is the specific medical examinee is acquired. In the data addition mode, the medical information including at least the age and the health data of the medical examinee of the medical examination, which is not acquired in the setting mode, is collected.

The health data evaluation unit 21 calculates the evaluation value obtained by evaluating the health data, which is a measurement value of a blood pressure, a blood glucose level, or the like measured in the medical examination, in stages according to the age based on a predetermined evaluation rule. For example, the evaluation is performed in four stages of scores A to D in order of a good evaluation. As the evaluation stage, the evaluation stages may be randomly integrated or parted such as a three-stage evaluation or a five-stage evaluation, as necessary. In the evaluation rule, it is preferable to use a criterion for a normal value range in the measurement value of each piece of health data or a criterion for determining an affection of each disease in a country in which the medical information processing apparatus 11 is operated or the medical institution to which the medical information processing apparatus 11 belongs. In the report creation mode, the evaluation value is calculated from the measurement result including the age and the health data of the specific medical examinee, and the age and the evaluation value are associated with each other.

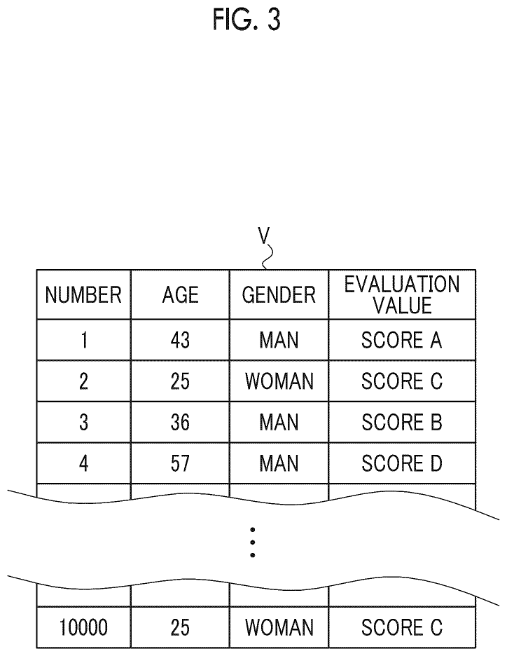

As shown in FIG. 3, the health data evaluation unit 21 creates evaluation value data V that is a medical information group in which the age and the evaluation value of each medical examinee are associated with each other in the report creation mode or the data addition mode. Each item of the evaluation value data V has at least a number or an ID for distinguishing or managing the age, the evaluation value, and the medical information of each medical examinee. In a case where the evaluation rule differs depending on the gender, it is preferable that the evaluation rule also includes gender information.

Further, an affection risk of the disease or a degree of severity of the disease may be calculated as the evaluation value from the health data having the measurement values of a plurality of items. In addition to a specific disease risk, one that evaluates a health state such as a body shape obtained from a body fat percentage or a muscle mass or a body mass index (BMI) obtained from a height and a weight may be used.

The distribution data creation unit 22 creates or updates the first distribution indicating the relationship between the age and the evaluation value, using the evaluation value data V. A graph consisting of an age axis in which one axis indicates the age and a score ratio axis (evaluation value ratio axis) in which the other axis indicates a ratio of the evaluation values is generated as the first distribution. As the first distribution, for example, a bar graph is used.

In the first distribution, an age spacing, which is a scale spacing of the age axis, is decided based on the number of evaluation values. Each scale on the age axis indicates the score ratio of the evaluation values in each age range based on the age spacing set in advance. The age spacing is adjusted such that there is no blank in the bar graph displayed as the first distribution. Unless otherwise specified, each score ratio is calculated from the evaluation value of the age spacing for every five years of age. In a case where there is a blank for every five years of age, the age spacing is adjusted, for example, for every ten years of age.

The region setting unit 23 sets the region in the first distribution based on the evaluation value and the age range in which the age axis is divided at a specific portion. Specifically, a plurality of regions are set from a region parting line that divides the age axis into a plurality of age ranges based on the specific portion set in advance, and the evaluation value. The upper limit number of region parting lines which can be set in the first distribution varies according to the number of scales of the age axis.

The specific portion is a portion where a change amount of a ratio occupied by a specific evaluation value is largest between the score ratios adjacent to each other on the age axis. The change amount is an increase amount or a decrease amount. A “decrease” in the ratio of the score evaluation value means that a ratio of any score evaluation value has a smaller value than the score ratio of the scale one level below. An “increase” in the ratio of the score evaluation value means that a ratio of any score evaluation value has a larger value than the score ratio of the scale one level below. It is preferable that the region to be set is parted into 16 regions as the maximum number of partings.

There are, in the score ratio of the first distribution, specific portions of a first portion where an amount of decrease in the score A is maximum, a second portion where an amount of increase in the score Cis maximum, and a third portion where an amount of increase in the score D is maximum. It is preferable that the set number of the region parting lines is preset and the region parting lines are set by applying the specific portions in order from the specific portion having the smallest number. Positions satisfying the first to third portions may overlap. Thus, a fourth portion and subsequent portions may be provided in which portions having the second largest increase or decrease amount in any score ratio are used as the portion. The specific portion may be provided before the region is set, or may be provided in real time at which an operator, such as a doctor, sets the region. Instead of applying each specific portion until the set number of region parting lines is satisfied, the number of portions to be applied may be determined in advance regardless of the number of region parting lines.

The specific portion may include a position where the score ratio is decreased or increased by a predetermined value or more, instead of or in addition to the maximum change amount. There may be a case where the change in the score ratio is small depending on the content of the collected medical information. Thus, distinguishment is made whether or not there is a significant difference in the change in the ratio occupied by the specific evaluation value between the adjacent score ratios on the age axis, which leads to more accurate data provision.

The sentence setting unit 24 sets the sentence in each region set by the region setting unit 23. A first sentence automatically set for each region, a second sentence set by a user input, and a third sentence combined from the first sentence and the second sentence are used properly as the sentences. It is preferable that the first sentence has a content, which corresponds to the measurement result of the user, that can be read from the measurement result and the first distribution, such as a tendency of the change in the score ratio according to the age or the setting portion of the region parting line. It is preferable that the second sentence has a content that encourages the user to take advice or pay attention from a doctor or the like according to the measurement result. A wording based on the fact that combining with the first sentence is performed may be input as the second sentence.

The sentence setting unit 24 combines the third sentence from the first sentence and the second sentence in response to completion of the input of the first sentence and the second sentence. In the combination, in addition to the combination of the input sentences, a new phrase is generated or an unnecessary phrase is deleted according to a specific keyword.

The automatic setting of the first sentence may be realized by a sentence generator (not shown) that automatically generates a sentence corresponding to each region by inference through machine learning. The sentence generator has a function of a learned model necessary for processing of generating the first sentence corresponding to each region in the first distribution, and executes sentence generation processing for each region. The sentence generator comprises a convolutional neural network (CNN), which is a computer algorithm consisting of a neural network that performs the machine learning, learns in advance data covering the score evaluation and the sentence describing the change in the score ratio according to the age, generates the first sentence in the set region, and outputs the first sentence in each region.

In the setting of the second sentence, the operator, such as a doctor, checks a first distribution 30 displayed on the display 13, and the second sentence is set by receiving a manual input by the operator, such as a doctor, via the user interface 14. Further, the first sentence may be corrected. Only one of the first sentence or the second sentence may be set in each region. In a case where the first sentence and the second sentence are set for the same region, it is preferable that the first sentence and the second sentence are combined into the third sentence to form one sentence. For the combination, the CNN or the like that learns a combination pattern of sentences and outputs one sentence from a plurality of input sentences may be employed.

The storage unit, such as a storage memory in the medical information processing apparatus 11, stores the first distribution after the sentence is set. After the storage, the setting mode ends. After the setting mode ends, the mode is switched to the report creation mode or the data addition mode.

In the report creation mode, the extraction unit 25 acquires the measurement result having the age and the evaluation value of the user who is the specific medical examinee from the health data evaluation unit 21, and extracts the second distribution to be listed in the report from the first distribution based on the specific region, which is a region that matches the measurement result discriminated by the specific region discrimination unit 26. The second distribution includes at least regions having different evaluation values at the same age spacing as the specific region. Further, the score ratio in a certain range before and after the age range of the specific region may be extracted as the second distribution regardless of the region set in the first distribution. It is preferable that the certain range is the score ratio for 1 or 2 scales before and after the age range of the specific region.

The provision data creation unit 28 creates the provision data, such as the report in which the second distribution, the sentence corresponding to the specific region, and the measurement result are displayed in the same display region. In the provision data, the measurement result for the specific region is displayed in a superimposed manner, or the specific region and other regions are displayed differently. Accordingly, the measurement result and the specific region including the measurement result can be viewed at a glance.

The created provision data is output to an external device such as an information terminal via the output control unit. The user browses the provision data through e-mail displayed on a screen of each external device or information technology (IT) content that can be browsed on the Internet. After the output, the report creation mode may be continued to further acquire the measurement result of the medical examinee for the creation of the report, the report creation mode may be ended to switch to the data addition mode, or the use of the medical information processing apparatus 11 may be ended. It is preferable that the created provision data is displayed on the display 13 and the operator, such as a doctor, checks the provision data.

As a first example, the creation of report will be described in which the medical information processing apparatus 11 performs the score evaluation on the affection risk of diabetes by using the measurement value regarding the blood glucose level of each medical examinee, which is measured in the health checkup from the medical device 12, and the age. In the first distribution, which is the distribution data, regions of 16 parts, which are four-parted with respect to the age axis by the region parting lines and four-parted for each score evaluation, are set.

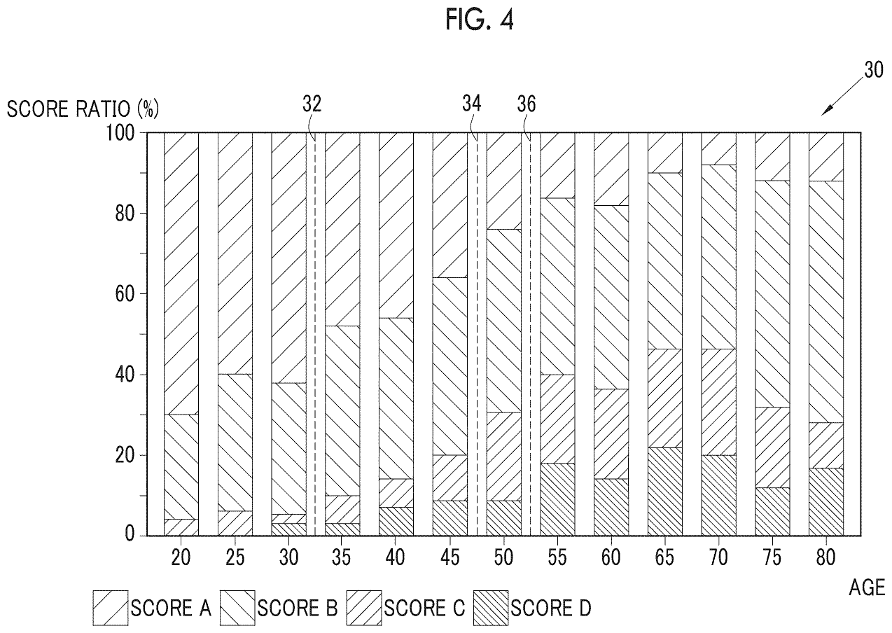

As shown in FIG. 4, the distribution data creation unit 22 creates the first distribution 30 as the distribution of the ages and the evaluation values obtained by performing the score evaluation on the health data. In the first distribution 30, the vertical axis is the ratio of the scores evaluated in four stages in units of percentages (%), and the horizontal axis is the age axis indicating the age range in which the ages are divided at a certain spacing in units of ages. The age axis having a scale of “20 years old” to “75 years old” is provided at the age spacing for every five years of age. In a case where there is the evaluation value of less than 20 years old, the evaluation value is included in the score ratio in the scale of “20 years old”, which is a minimum value of the scale. In a case where there is the evaluation value of 80 years old or older, the evaluation value is included in the score ratio in the scale of “75 years old”, which is a maximum value of the scale. In a case where the number of evaluation values on the scale of the minimum value or the maximum value is insufficient, a scale numerical value of an end part of the age range is rounded. It is preferable to set a minimum age in each age range as a value of each scale. In a case of the age spacing for every five years of age, the scale of the horizontal axis of “20” includes the ages of 20 years old to 24 years old. In the report creation, according to the evaluation of the four stages of scores A to D, the scores A to D will be respectively described as “no abnormality”, “attention required”, “improvement required”, and “medical treatment required”.

The score A is a state of “no abnormality”, in which the evaluation result is obtained in which the evaluation value is within the normal value range and the affection risk is lowest, whereas the measurement value tends to deteriorate with aging, which decreases the ratio occupied by the score A. Thus, it is preferable to create the sentence of the score A with a content that emphasizes the importance of being the score A or a content that is desired to be maintained as a current value since the value is a good measurement value, as the age is higher. This makes it possible to encourage the maintenance of motivation.

The score B is a state of “attention required”, in which the affection risk is relatively low since the evaluation value is within the normal value range, but the affection risk is higher than that in the score A. Thus, there is room for improvement in the measurement value. The improvement of the affection risk is called while recommending that the normal value range is maintained. This makes it possible to encourage the maintenance of motivation or the improvement of the measurement value.

The score C is a state of “improvement required”, in which the evaluation value is out of the normal value range, but the affection risk is higher than that of the score B. Thus, the improvement of the risk can be expected by improving lifestyle habits. Advice on improving the lifestyle habits or checkup at a specialized medical institution is suggested. This makes it possible to prevent further deterioration and promote the improvement of the health state. Further, early medical treatment is encouraged.

The score D is a state of “medical treatment required”, in which the evaluation value is out of the normal value range and the affection risk is the highest. There is a sufficient possibility of being suffered from the disease to be evaluated. Thus, an instruction to improve the lifestyle habits is made or checkup at a specialized medical institution is checked. This makes it possible to encourage detailed grasping of the health state by a detailed examination or the like, promotion of the health state, or medical treatment.

In the creation or display of the distribution, in a case of a monochromatic image such as a black-and-white image, it is preferable to distinguish each score by shading. A density of the region is lighter as the score is better, the score A being the lightest region and the score D being the darkest region. In a case of a color image, it is preferable to display each score in an easily distinguishable color.

In “15”, which is a value at an end of the scale, the evaluation value of 14 years old or younger is included in addition to a range of 15 years old to 19 years old. That is, in “15”, the score ratio of the evaluation values of 19 years old or younger is displayed. Similarly, in “75”, which is a value at the other end of the scale, the evaluation value of 80 years old or older is included in addition to a range of 75 years old to 79 years old. That is, in “75”, the score ratio of the evaluation values of 80 years old or older is displayed.

In the first distribution 30 transmitted to the region setting unit 23, the region parting line that divides the age ranges is set based on each predetermined portion. Here, with a maximum decrease amount of the score A as the first portion, a maximum increase amount of the score C as the second portion, and the maximum increase amount of the score D as the third portion, a first region parting line 32, a second region parting line 34, and a third region parting line 36 are set in order (refer to FIG. 4).

The first portion is satisfied between “30 years old” and “35 years old” where the score A decreases by 15 points from 63% to 48%. Thus, the first region parting line 32 is set between the scales of “30 years old” and “35 years old”. The second portion is satisfied between “45 years old” and “50 years old” where the score C increases by 11 points from 11% to 22%. Thus, the second region parting line 34 is set between the scales of “45 years old” and “50 years old”. The third portion is satisfied between “50 years old” and “55 years old” where the score D increases by 9 points from 9% to 18%. Thus, the third region parting line 36 is set between the scales of “50 years old” and “55 years old”.

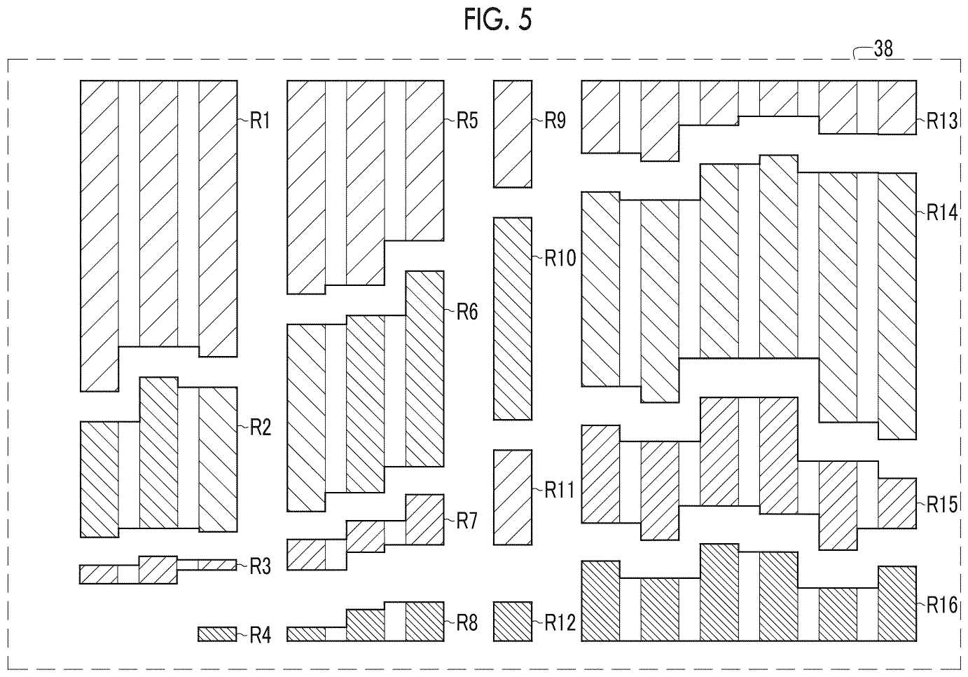

As shown in FIG. 5, each region of the first distribution 30 set by the region setting unit 23 is stored in the storage unit such as a storage memory together with the first distribution 30 as a region data group 38. Respective regions in the region data group 38 are set as a region R1 to a region R16 in descending order of the age ranges in the first distribution 30 and in descending order of the scores in a case where the age ranges are the same in the first distribution 30. A minimum configuration of the region is a region indicating the evaluation value in one age range, such as the regions R4 and R9 to R12. Names of a first region to a sixteenth region are used properly for the region R1 to the region R16.

In the age range in which both ends of the age scale are 15 years old and 30 years old, that is, 34 years old or younger, a region of the score A is set as the region R1, a region of the score B is set as the region R2, a region of the score C is set as the region R3, and a region of the score D is set as the region R4. In the age range in which both ends of the age scale are 35 years old and 50 years old, that is, 35 to 54 years old, a region of the score A is set as the region R5, a region of the score B is set as the region R6, a region of the score C is set as the region R7, and a region of the score D is set as the region R8. In the age range in which the age scale is only 50 years old, that is, 50 years old to 54 years old, a region of the score A is set as the region R9, a region of the score B is set as the region R10, a region of the score C is set as the region R11, and a region of the score D is set as the region R12. In the age range in which both ends of the age scale are 55 years old and 80 years old, that is, 55 years old or older, a region of the score A is set as the region R13, a region of the score B is set as the region R14, a region of the score C is set as the region R15, and a region of the score D is set as the region R16.

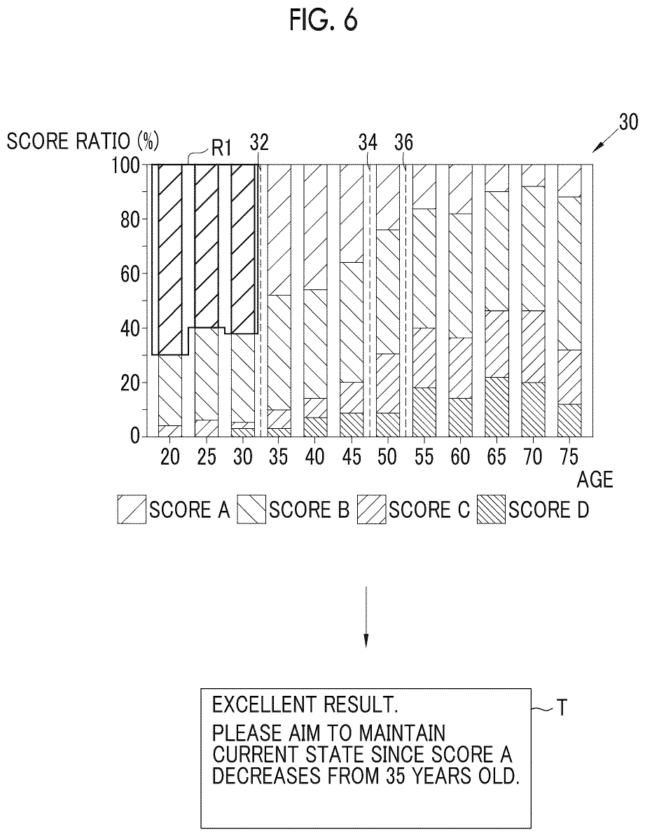

As shown in FIGS. 6 to 10, the sentence setting unit 24 generates the sentence to be listed in the report for each parted region. The sentence is input by the operator, such as a doctor, and stored in association with each region of the region data group 38.

As shown in FIG. 6, in a case of the score A in the region R1, that is, 34 years or younger, a sentence T (for example, “Excellent result”) that praises the lifestyle habits or the measurement result of the medical examinee or the sentence T (for example, “Please aim to maintain current state”) that encourages the maintenance of the current state is generated.

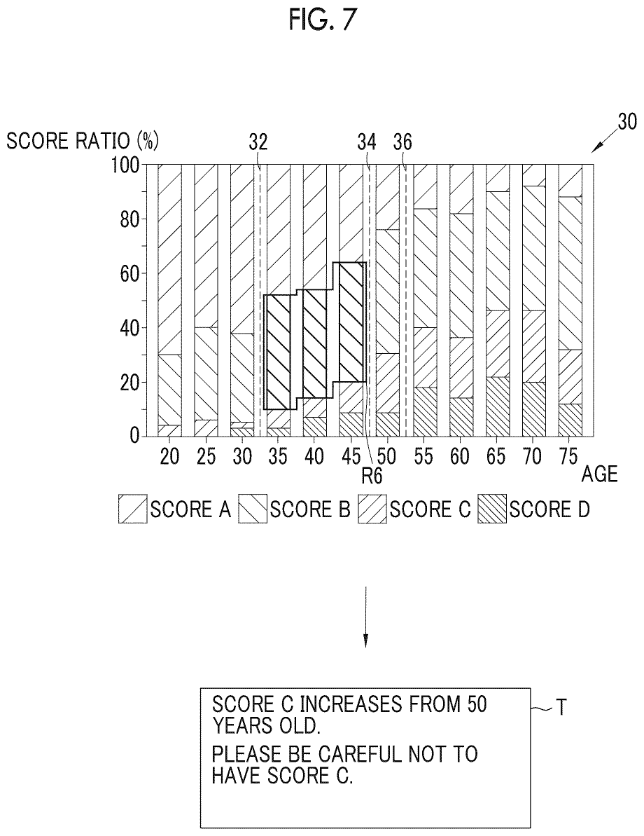

As shown in FIG. 7, in a case of the score B in the region R6, that is, between 35 years old to 49 years old, the sentence T (for example, “Be careful”) that calls attention to the lifestyle habits or the measurement result is created.

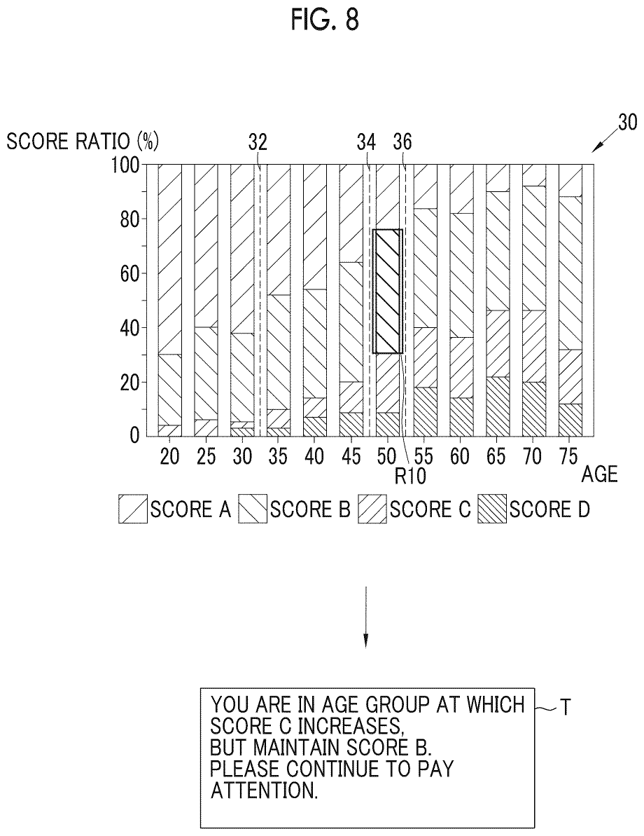

As shown in FIG. 8, in a case of the score B in the region R10, that is, between 50 years old to 54 years old, since a young-aged side is parted by the second portion, the sentence that notifies the medical examinee of the increase in the score C is created. Further, since 50 years old to 54 years old are the age groups at which the score D is concerned to increase in the future, the sentence T (for example, “Please continue to pay attention”) that encourages continued attention to the lifestyle habits or the measurement result is created.

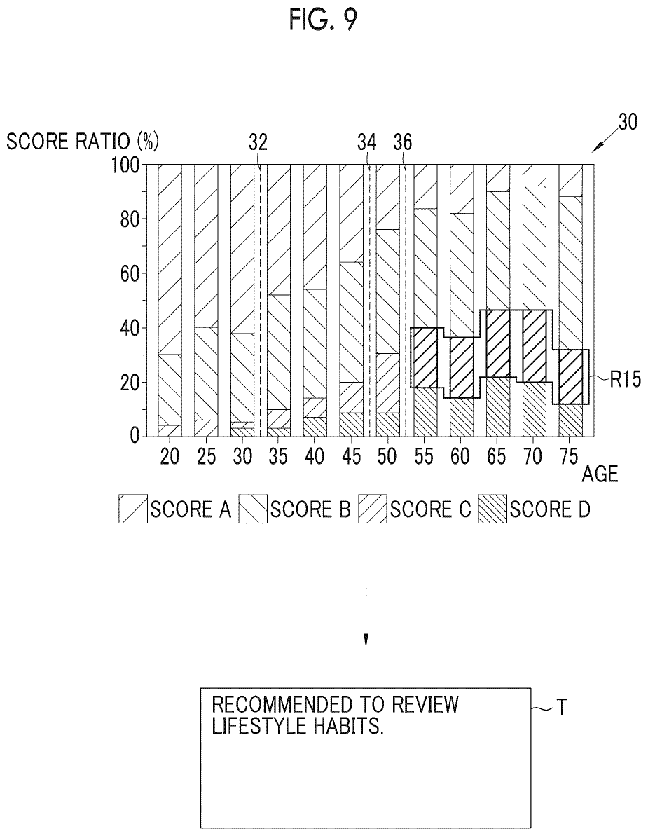

As shown in FIG. 9, in a case of the score C in the region R15, that is, 55 years old or older, the sentence T that encourages improvement (for example, “Recommended to review lifestyle habits”) or the sentence that encourages a further medical examination such as a detailed examination or a medical examination by a medical specialist is input.

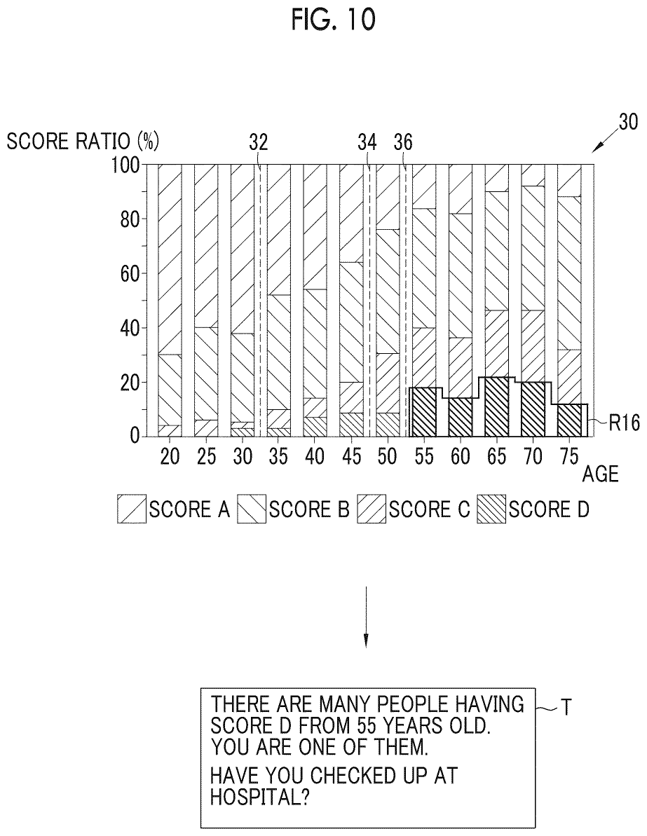

As shown in FIG. 10, in a case of the score D is in the region R16, that is, 55 years old or older, the sentence that presents a medical treatment option or the sentence T that encourages the medical treatment (for example, “Have you checked up at hospital?”) is input. Further, the fact that 55 years old or older are the age groups at which the score D increases is emphasized.

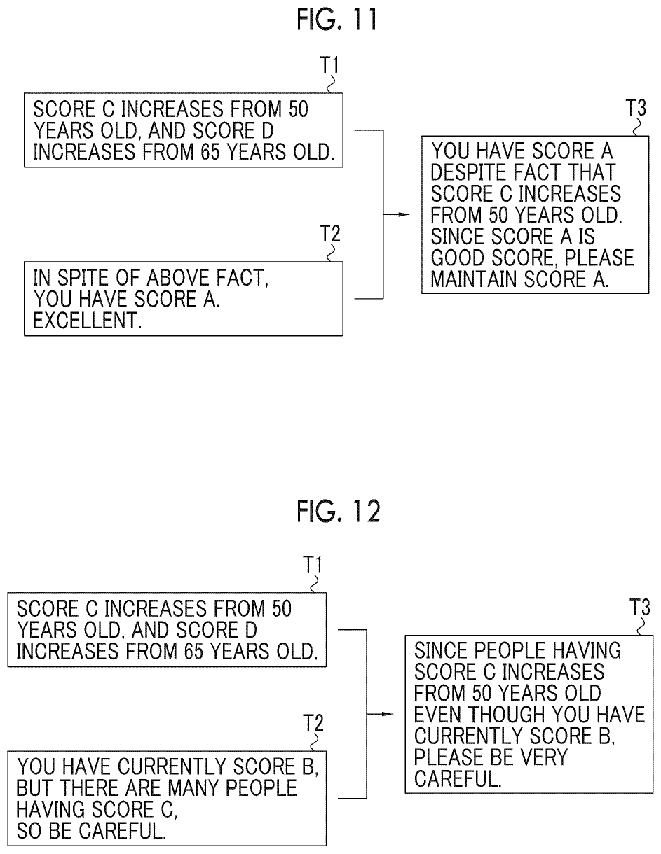

As shown in FIG. 11, a first sentence T1 and a second sentence T2 are used to perform a combination of a third sentence T3. For example, regarding the generation of the third sentence T3 of the region R9, in the region R9 which is the score A at 50 years old to 54 years old, the sentence generator generates the first sentence T1 of “Score C increases from 50 years old, and score D increases from 65 years old”. In a case where the doctor checks the content of the first sentence T1 and sets the second sentence T2 of “In spite of the above fact, you have score A. Excellent.”, which is input via a user interface, in the generation of the third sentence T3, a portion related to “score D”, which is discriminated to be unnecessary, is deleted, and a wording of “Please maintain score A.” is added regarding “score A”.

As shown in FIG. 12, for example, regarding the generation of the third sentence T3 of the region R10, in the region R9 with the score B at 50 years old to 54 years old, the sentence generator generates the first sentence T1 of “Score C increases from 50 years old, and score D increases from 65 years old”. In a case where the doctor checks the content of the first sentence T1 and sets the second sentence T2 of “You have currently score B, but there are many people having the score C, so be careful.”, which is input via a user interface, in the generation of the third sentence T3, a portion related to “score D”, which is discriminated to be unnecessary, is deleted, and the wording changes from “Be careful” to “Please be very careful” regarding “score B”.

The storage memory stores the first distribution 30 parted into the regions R1 to R16 and data of comments C1 to C16 corresponding to each region. The medical information processing apparatus 11 that has performed the region setting and the comment setting switches the mode to the report creation mode to create the report for the acquired measurement result.

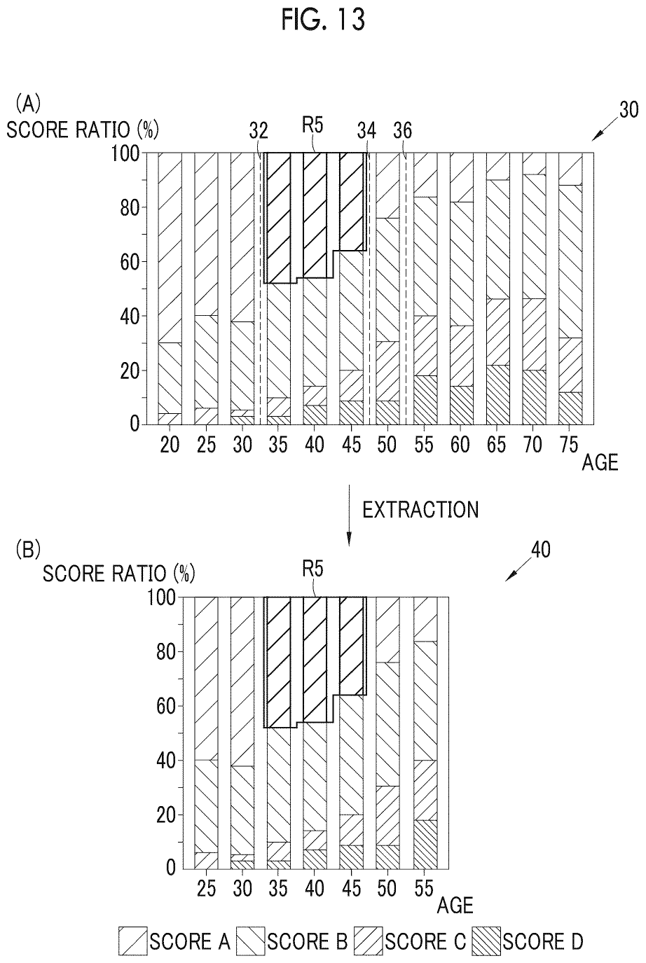

As shown in FIGS. 13A and 13B, in the report creation mode, the specific region is discriminated by the specific region discrimination unit 26 in the extraction unit 25. In the measurement result in which the age is 43 years old and the evaluation value is the score A, the region R5 is the specific region in the first distribution 30 (refer to FIG. 13A), and a second distribution 40 (refer to FIG. 13B) is extracted based on the region R5. The score ratio of the regions R6 to R8, which are regions having the same age spacing as the specific region, and the score ratio for every consecutive two scales before and after the age spacing are set as an extraction range. That is, the score ratio data in the range of 35 years old to 49 years old including the specific region and the score ratio data in the ranges of consecutive age groups of 25 years old to 34 years old and 50 years old to 59 years old are extracted as the second distribution 40.

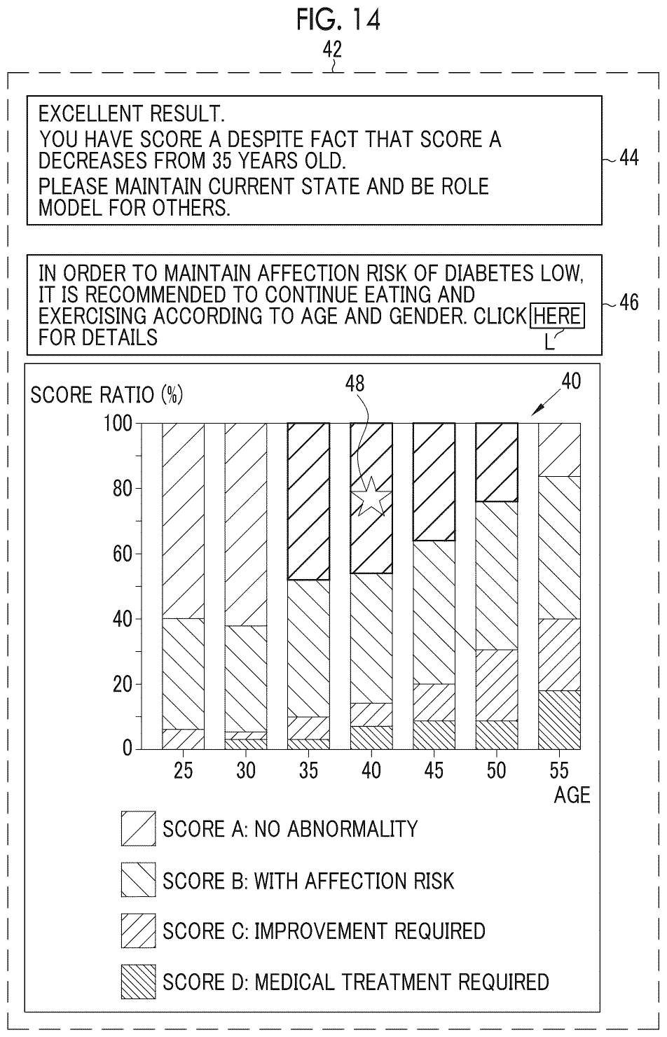

As shown in FIG. 14, the provision data creation unit 28 creates a report 42. In the report 42, the second distribution 40, a sentence field 44 that displays any one of the sentences T1 to T3 corresponding to the specific region, and a flag 48 indicating a position of the measurement result are superimposed on the specific region. The flag 48 in the first example indicates the measurement result in which the age is 43 years old and the score is A. Thus, the flag 48 is displayed in a superimposed manner in a region on the age scale of “40 years old” on the region R5. The sentence corresponding to the specific region is a sentence stored in the storage unit in association with the regions R1 to R16 discriminated as the specific region.

Further, sizes of the second distribution 40 and the sentence, which are displayed in report 42, are changed according to the specific region. The size based on the specific region, a sentence amount that varies according to the score and the age, or the size of the second distribution 40 is adjusted to facilitate the check by the user. For example, since the score is A in the specific region of the region R5, the sentence to the user is shorter than other scores. In that case, the sentence field 44 is smaller according to the sentence amount. On the other hand, since the score is D in the specific region of the region R16, the sentence to the user is a long sentence. In that case, the sentence field 44 is displayed large, and the second distribution 40 is displayed small according to the change of the sentence field 44.

The report 42 may be provided with a second sentence field 46 for displaying information provided to the user, according to an item other than the second distribution, which is different from the sentence field 44 displayed according to the specific region. It is preferable that the second sentence field 46 displays information related to an item measured by a medical examination which is health data, advice based on biological information of the user including the gender, or the like. Further, in a case where the report 42 is provided to the user as e-mail or IT content, a link L may be provided in which a reference uniform resource locator (URL) is embedded in the second sentence field 46.

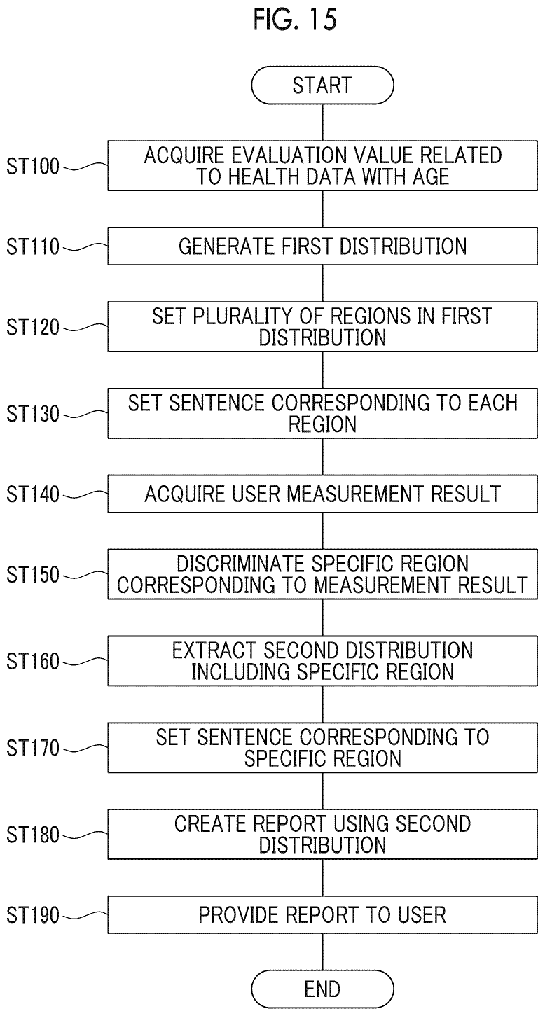

A flow of a series of operations in the report creation of the medical information processing apparatus 11 according to the present embodiment will be described with reference to a flowchart shown in FIG. 15. The medical information processing apparatus 11 receives, in the setting mode, an input of the medical information having the age and the health data from the medical device 12, and acquires the evaluation value calculated by the health data evaluation unit 21 together with the age (step ST100). The first distribution 30 indicating the distribution of the ages and the evaluation values is generated based on the age and the evaluation value of the health data (step ST110). The region parting line is set in the first distribution 30 according to the specific portion set in advance, and the plurality of regions in the first distribution are set based on the region parting line and the evaluation value (step ST120). For each region parted in the first distribution 30, the sentence for report listing, in which the relationship between the age and the evaluation value is described, is set and stored (step ST130). After the storage, the setting mode ends.

After the end of the setting mode, the mode is switched to the report creation mode, and the measurement result is acquired for which the evaluation value can be calculated, which is the result of the medical examination of the specific user (step ST140). The specific region discrimination unit 26 discriminates the specific region, which corresponds to the age and the evaluation value in the measurement result, from each region of the first distribution 30 (step ST150). The second distribution 40 including at least the region on the same scale as the discriminated specific region is extracted from the first distribution 30 (step ST160). The sentence corresponding to the specific region is set from the stored sentence corresponding to each region (step ST170). The report that displays the extracted second distribution 40, the sentence corresponding to the specific region, and the measurement result is created (step ST180). The created report is provided to the user who is the medical examinee as e-mail or IT content (step ST190).

In the display of the first distribution 30 and the second distribution 40, a portion where the score ratio is maximum in each evaluation value in the distribution data may be displayed differently from the others, separately from the specific region, to attract attention. Further, with addition of a measurement date and time to the medical information acquired from the medical device 12, the distribution data may be created for each measurement year such that transition of a proportion in which the health data in each age range is within the normal value range can be checked.

As a second example, a pattern in which data is added to create the report will be described. The medical information processing apparatus 11 switches the mode to the data addition mode after the setting mode or the report creation mode ends to receive addition of the medical information including the age and health data from the medical device 12. Since the score ratio and the position of each region parting line are changed by the addition, the region setting in the first distribution is updated. After the resetting of the region, the mode is switched to the report creation mode and the report is created for the measurement result. For others, each setting and the report creation are performed in the same manner as in the first example, and thus the description thereof will be omitted.

The distribution data creation unit 22 updates the distribution whose type matches the additionally input health data. It is preferable to perform display indicating a portion of interest in the first distribution updated based on the data addition. The portion of interest is a region parting line or a region that is reset by the update. In a case where the region parting line is set as the portion of interest, an indicator, such as an arrow, indicating the position of the region parting line after movement, is displayed. In a case where the region is set as the portion of interest, display that can be discriminated at a glance is performed, such as coloring the region whose range has been changed.

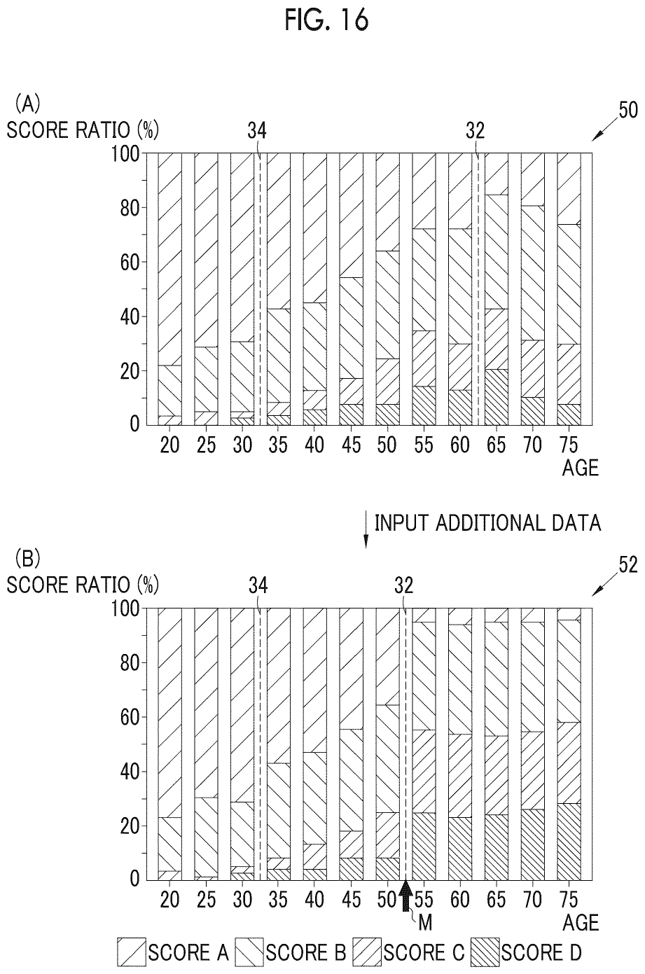

As shown in FIGS. 16A and 16B, in a case where the same type of health data and the medical information including the age of the medical examinee are added, the score ratio in a first distribution 50 changes. Thus, a form of the region parting line and the region is updated as in a first distribution 52. The first distribution 50 in FIG. 16A is a distribution of the ages and the evaluation values of the health data, which is different from the first distribution 30 in the first example. The age axis has a scale from “20 years old” to “75 years old” with the age range for every five years of age as one scale. Further, the set number of region parting lines is set to “two”. The first region parting line 32 is set between “60 years old” and “65 years old”, and the first to third portions are common. The second region parting line 34 is set between “30 years old” and “35 years old”, which is a position where the fourth portion with the second largest decrease amount of the ratio of the score A is satisfied. Regions R1 to R12 of 12 parts, which are three-parted obtained by parting the age range by two region parting lines and four-parted for each score evaluation, are decided.

In the first distribution 52 of FIG. 16B, the score ratio is changed from the first distribution 50 by the input of the additional data, and the region parting line is reset. The region resetting is performed based on the changed region parting lines. In the first distribution 52, since a position satisfying the first portion to the third portion is between “50 years old” and “55 years old”, the first region parting line 32 is moved between “50 years old” and “55 years old” on the age axis. Further, an arrow M indicating the movement is displayed. Since the position satisfying the fourth portion is between “30 years old” and “35 years old”, the second region parting line 34 is set at the same position as the first distribution 50 before the update.

For each region after the update, a change of the corresponding sentence is received, and the sentence is reset. On the other hand, region characteristics of a region in which the age range of the region after the update is not changed, as compared with the region before the update, and the change amount of the score ratio is within the certain range are discriminated to be common. For the region having common region characteristics, that is, the same feature as before the update, the sentence set before the update is reused. This makes it possible to reduce the time and effort for resetting the sentence and perform efficient update.

In the first distribution 52, similarly to the first distribution 50, the regions R1 to R12 of 12 parts, which are three-parted obtained by parting the age range by two region parting lines and four-parted for each score evaluation, are reset. In the first distribution 52, each region of the scores A to D in the age less than “35 years old” has the same region characteristics as the region R1 to the region R4 in the first distribution 50. Therefore, the sentences corresponding to the regions R1 to R4 in the first distribution 50 before the update are reused.

Further, in a case where a sufficient amount of the medical information of the medical examinee who is 80 years old or older is acquired in the additional data, the scale of “80 years old” on the age axis and a bar graph may be added. In a case where a sufficient amount of the medical information of the medical examinee who is less than 20 years old is acquired, the scale of “15 years old” on the age axis and a bar graph may be added. The sufficient amount thereof is preferably at least 10.

### Second Embodiment

In the first embodiment, the first distribution is generated in which the score ratio of the age range for every five years of age on the age axis is calculated. However, the medical information acquired from the medical device 12 may be insufficient in some age groups. Thus, the setting mode in a case where a large amount of acquired medical information has blanks in some age groups will be described. Other contents are the same as those of the first embodiment, and thus the description thereof will be omitted.

In a second embodiment, the age range is detected in which the number of evaluation values is insufficient, such as less than a certain number, and the age spacing or a value of an edge scale is adjusted. The scale of the age axis is adjusted to integrate the evaluation values in consecutive scales. The detection is made that the number of evaluation values is insufficient in a case where the number of evaluation values is smaller than a predetermined minimum number of cases in each age range. The minimum number of cases may be 10, but may be calculated using an average age or a standard deviation. In the calculation of the minimum number of cases, in a case where the number of evaluation values in each age range is set as en, the average age is set as av, and the standard deviation is set as sd, it is preferable to satisfy the following Expression (1).

en≥av−3sd (1)

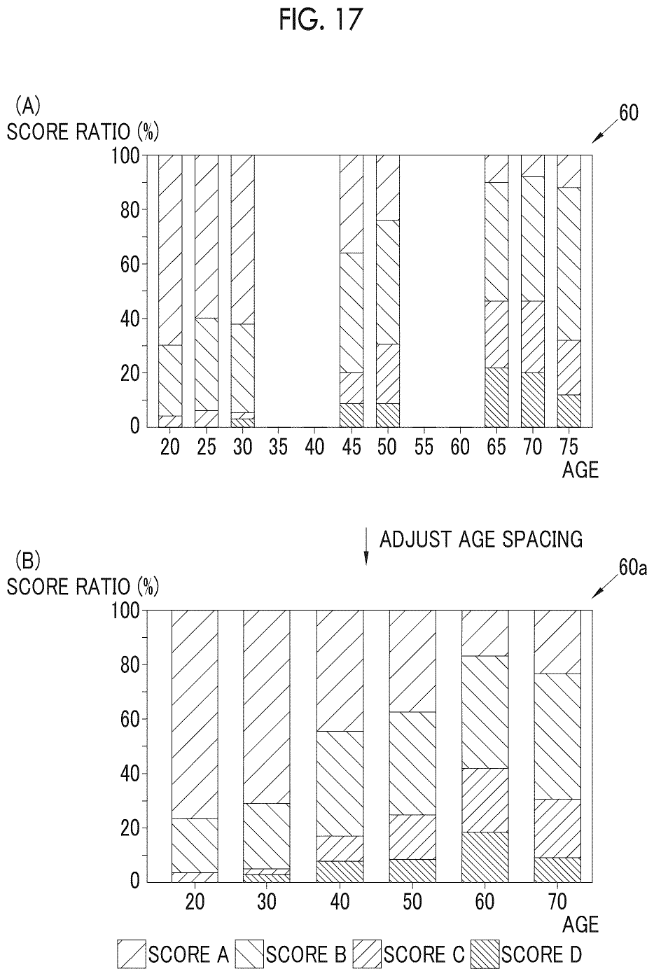

As shown in FIGS. 17A and 17B, in a case where the number of the evaluation values of a specific age group that is not the end part of the age range in a first distribution 60 is insufficient, the age spacing is adjusted in accordance with the insufficient age range. In the first distribution 60 which is the age spacing for every five years of age in FIG. 17A, the evaluation values in the age ranges of 35 years old to 44 years old and 55 years old to 64 years old are insufficient, and there are blanks in the bar graph. In the first distribution 60, since the score ratio can be calculated at the scale next to the scale in which the bar graph is blank, there are a sufficient number of evaluation values, and two scales for every five years of age can be integrated into the one scale for every 10 years of age. In a first distribution 60a of FIG. 17B, with the integration of the blank bar graph and the bar graph having the sufficient evaluation value, the bar graph is displayed at the age spacing for every 10 years of age, and the blanks are eliminated. With the integration, the age axis having the scale with five-year spacings is changed to 10-year spacings.

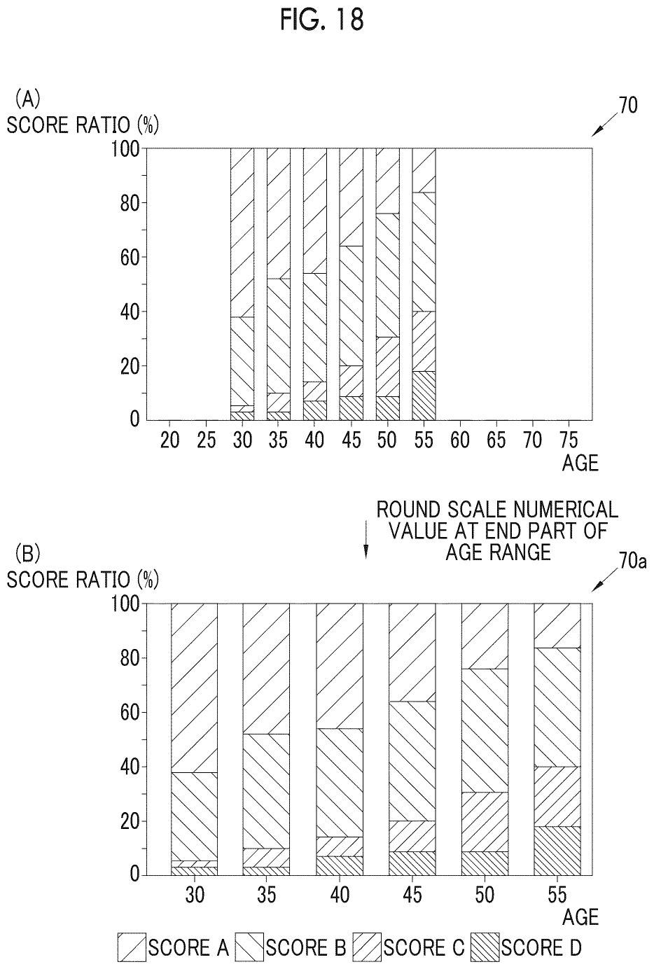

As shown in FIGS. 18A and 18B, in a case where the number of evaluation values at the end part of the age range is insufficient in a first distribution 70, the scale numerical value at the end part of the age range is rounded. In the first distribution 70 having the age axis in which a maximum value in FIG. 18A is the scale of “75 years old” and a minimum value therein is the scale of “20 years old”, the medical information of the medical examinees who are less than 30 years old and 60 years old or older is insufficient, and there are blanks in the bar graph. Since the score ratio can be calculated in the age range from 30 years old to 59 years old, the maximum value and the minimum value of the scales of the age axis are adjusted. In a first distribution 70a in FIG. 18B, in the first distribution 70a obtained by rounding the numerical value of the scale at the end part of the age axis, a maximum value of the scale on the age axis is “55 years old”, a minimum value thereof is “30 years old”. Therefore, the evaluation value of the medical examinee who is less than 34 years is integrated into the bar graph of the score ratio of “30 years old”, and the evaluation value of the medical examinee who is 55 years old or older is integrated into the bar graph of the score ratio of “55 years old”.

In the second embodiment, since the number of scales is reduced after the adjustment of the numerical value on the age axis by the adjustment or rounding, the region of each score ratio can be displayed with a width larger than that before the change. Further, it is preferable to set an upper limit number of region parting lines set in the report creation mode to “two”. In a case where the number of scales is equal to or less than a certain number as in the first distribution 60a or the first distribution 70a, the extraction after the discrimination of the specific region may not be performed, and the first distribution may be listed in the report 42. In the data addition mode, in a case where the medical information in the blank age group is sufficiently obtained, it is preferable to change the scale on the age axis in accordance with the additional data in a case of the distribution update.

In each embodiment, a hardware structure of the processing unit that executes various pieces of processing, such as the central control unit, the input reception unit, the output control unit, the data acquisition unit 20, the health data evaluation unit 21, the distribution data creation unit 22, the region setting unit 23, the sentence setting unit 24, the extraction unit 25, and the provision data creation unit 28, is various processors as shown below. The various processors include a central processing unit (CPU) that is a general-purpose processor which functions as various processing units by executing software (programs), a programmable logic device (PLD) that is a processor of which a circuit configuration can be changed after manufacturing, such as a field programmable gate array (FPGA), a dedicated electrical circuit that is a processor having a circuit configuration exclusively designed to execute various types of processing, and the like.

One processing unit may be composed of one of these various processors or a combination of two or more processors having the same type or different types (for example, combination of a plurality of FPGAs, or CPU and FPGA). Further, a plurality of processing units may be composed of one processor. As an example in which the plurality of processing units are composed of one processor, first, as represented by a computer such as a client and a server, a form in which one processor is composed of a combination of one or more CPUs and software and the processor functions as the plurality of processing units is possible. Second, as represented by a system on chip (SoC) and the like, a form of using a processor that implements functions of the entire system including the plurality of processing units in one integrated circuit (IC) chip is possible. As described above, the various processing units are configured by using one or more various processors as the hardware structure.

Furthermore, the hardware structure of the various processors is more specifically an electric circuit (circuitry) having a form in which circuit elements such as semiconductor elements are combined. Further, a hardware structure of the storage unit is a storage device such as a hard disk drive (HDD) or a solid state drive (SSD). Further, from the above description, the medical information processing apparatus according to the following Supplementary Notes 1 to 14 can be grasped.

### Supplementary Note 1

A medical information processing apparatus comprising:
a processor,
wherein the processor is configured to:
generate a first distribution based on an age and an evaluation value related to health data;
set a plurality of regions and a sentence corresponding to each of the regions in the first distribution;
acquire a measurement result including the age and the evaluation value of a user;
extract a second distribution including a specific region, which is the region corresponding to the measurement result, from the first distribution; and
display the second distribution, the sentence corresponding to the specific region, and the measurement result in the same display region.

### Supplementary Note 2

The medical information processing apparatus according to Supplementary Note 1,
wherein the processor is configured to:
calculate the evaluation value from the health data evaluated in stages according to the age; and
generate, as the first distribution, a graph in which one axis is an age axis indicating the age and the other axis is a score ratio axis indicating a ratio of the evaluation value.

### Supplementary Note 3

The medical information processing apparatus according to Supplementary Note 2,
wherein the processor is configured to:
decide an age spacing, which is a scale spacing on the age axis, based on the number of the evaluation values in the first distribution.

### Supplementary Note 4

The medical information processing apparatus according to Supplementary Note 2 or 3,
wherein the processor is configured to:
set the region in the first distribution based on the evaluation value and an age range in which the age axis is divided at a specific portion.

### Supplementary Note 5

The medical information processing apparatus according to Supplementary Note 4,
wherein the specific portion is a portion where an amount of change in a specific evaluation value is largest in ratios between two consecutive evaluation values.

### Supplementary Note 6

The medical information processing apparatus according to any one of Supplementary Notes 1 to 5,
wherein the processor is configured to:
display the display region in which the measurement result is displayed on the specific region in a superimposed manner or the specific region is displayed differently from another region.

### Supplementary Note 7

The medical information processing apparatus according to any one of Supplementary Notes 1 to 6,
wherein the processor is configured to:
decide the sentence and a size of the second distribution to be displayed in the display region, based on the specific region.

### Supplementary Note 8

The medical information processing apparatus according to any one of Supplementary Notes 1 to 7,
wherein the processor is configured to:
receive addition of the age and the health data; and update the setting of each of the regions in the first distribution based on the addition.

### Supplementary Note 9

The medical information processing apparatus according to Supplementary Note 8,
wherein the processor is configured to:
perform display indicating a portion of interest in the first distribution updated based on the addition.

### Supplementary Note 10

The medical information processing apparatus according to Supplementary Note 8 or 9,
wherein the processor is configured to:
reset the sentence corresponding to each of the regions updated based on the addition.

### Supplementary Note 11

The medical information processing apparatus according to Supplementary Note 10,
wherein the processor is configured to:
reuse, for the region having the same feature as before update, the sentence before the update in the resetting.

### Supplementary Note 12

The medical information processing apparatus according to any one of Supplementary Notes 3 to 5,
wherein the processor is configured to:
adjust, in the first distribution, the age spacing in a case where the number of the evaluation values is less than a certain number.

### Supplementary Note 13

The medical information processing apparatus according to any one of Supplementary Notes 3 to 5,
wherein the processor is configured to:
round, in a case where the number of the evaluation values in a scale of an end portion on the age axis is less than a certain number, a numerical value of the scale of the end portion in the first distribution.

### Supplementary Note 14

The medical information processing apparatus according to any one of Supplementary Notes 1 to 13,
wherein the processor is configured to:
properly use, in the setting of the sentence, a first sentence automatically set for each specific region, a second sentence set by a user input, and a third sentence combined from the first sentence and the second sentence.

### EXPLANATION OF REFERENCES

10: medical information processing system
11: medical information processing apparatus
12: medical device
13: display
14: user interface
20: data acquisition unit
21: health data evaluation unit
22: distribution data creation unit
23: region setting unit
24: sentence setting unit
25: extraction unit
26: specific region discrimination unit
28: provision data creation unit
30: first distribution
32: first region parting line
34: second region parting line
36: third region parting line
38: region data group
40: second distribution
42: report
44: sentence field
46: second sentence field
48: flag
50: first distribution
52: first distribution
60: first distribution
60a: first distribution
70: first distribution
70a: first distribution
L: link
M: arrow
R1 to R16: region
ST100 to ST190: step
T: sentence
T1: first sentence
T2: second sentence
T3: third sentence
V: evaluation value data

What is claimed is:

1. A medical information processing apparatus comprising:
a processor,
wherein the processor is configured to:
generate a first distribution based on an age and an evaluation value related to health data;
set a plurality of regions and a sentence corresponding to each of the regions in the first distribution;
acquire a measurement result including the age and the evaluation value of a user;
extract a second distribution including a specific region, which is the region corresponding to the measurement result, from the first distribution; and
display the second distribution, the sentence corresponding to the specific region, and the measurement result in the same display region.

2. The medical information processing apparatus according to claim 1,
wherein the processor is configured to:
calculate the evaluation value from the health data evaluated in stages according to the age; and
generate, as the first distribution, a graph in which one axis is an age axis indicating the age and the other axis is a score ratio axis indicating a ratio of the evaluation value.

3. The medical information processing apparatus according to claim 2,
wherein the processor is configured to:
decide an age spacing, which is a scale spacing on the age axis, based on the number of the evaluation values in the first distribution.

4. The medical information processing apparatus according to claim 3,
wherein the processor is configured to:
set the region in the first distribution based on the evaluation value and an age range in which the age axis is divided at a specific portion.

5. The medical information processing apparatus according to claim 4,
wherein the specific portion is a portion where an amount of change in a specific evaluation value is largest in ratios between two consecutive evaluation values.

6. The medical information processing apparatus according to claim 1,
wherein the processor is configured to:
display the display region in which the measurement result is displayed on the specific region in a superimposed manner or the specific region is displayed differently from another region.

7. The medical information processing apparatus according to claim 1,
wherein the processor is configured to:
decide the sentence and a size of the second distribution to be displayed in the display region, based on the specific region.

8. The medical information processing apparatus according to claim 1,
wherein the processor is configured to:
receive addition of the age and the health data; and
update the setting of each of the regions in the first distribution based on the addition.

9. The medical information processing apparatus according to claim 8,
wherein the processor is configured to:
perform display indicating a portion of interest in the first distribution updated based on the addition.

10. The medical information processing apparatus according to claim 8,
wherein the processor is configured to:
reset the sentence corresponding to each of the regions updated based on the addition.

11. The medical information processing apparatus according to claim 10,
wherein the processor is configured to:
reuse, for the region having the same feature as before update, the sentence before the update in the resetting of the sentence.

12. The medical information processing apparatus according to claim 3,
wherein the processor is configured to:
adjust, in the first distribution, the age spacing in a case where the number of the evaluation values is less than a certain number.

13. The medical information processing apparatus according to claim 3,
wherein the processor is configured to:
round, in a case where the number of the evaluation values in a scale of an end portion on the age axis is less than a certain number, a numerical value of the scale of the end portion in the first distribution.

14. The medical information processing apparatus according to claim 1,
wherein the processor is configured to:
properly use, in the setting of the sentence, a first sentence automatically set for each specific region, a second sentence set by a user input, and a third sentence combined from the first sentence and the second sentence.

15. A non-transitory computer readable medium for storing a computer-executable program, the computer-executable program causing a computer to execute:
a function of generating a first distribution based on an age and an evaluation value related to health data;
a function of setting a plurality of regions and a sentence corresponding to each of the regions in the first distribution;
a function of acquiring a measurement result including the age and the evaluation value of a user;
a function of extracting a second distribution including a specific region, which is the region corresponding to the measurement result, from the first distribution; and
a function of displaying the second distribution, the sentence corresponding to the specific region, and the measurement result in the same display region.
